(19) United States
(12) Patent Application Publication
(10) Pub. No.: US 2018/0289510 A1
(43) Pub. Date: Oct. 11, 2018

(54) GYROSCOPICALLY CONTROLLED BALANCE PROSTHETIC

(71) Applicant: Worcester Polytechnic Institute, Worcester, MA (US)

(72) Inventors: Mitchell K. Muller, Worcester, MA (US); Marko B. Popovic, Cambridge, MA (US)

(21) Appl. No.: 15/948,270

(22) Filed: Apr. 9, 2018

Related U.S. Application Data
(60) Provisional application No. 62/482,780, filed on Apr. 7, 2017.

(51) Int. Cl. A61F 2/58; A61F 2/68; A61F 2/76

(52) U.S. Cl. CPC A61F 2/581; A61F 2002/546; A61F 2/76; A61F 2/68

(57) ABSTRACT

A shoulder prosthesis restores the complex dynamics of the arm for whole-arm amputees. While most prosthesis for arm amputees focus on restoring the user's capabilities for dexterous manipulation, the disclosed prosthesis remains affixed to the shoulder and exerts a moment on the user's trunk similar to that of the arm during walking for dynamic motion assistance. The prosthesis includes a rotating, gimbaled mass oriented based on gait and stride for emulating forces that would have been provided by the amputee arm. The size, ease of use, and relatively low cost manufacture of the proposed device makes it an attractive complement or alternative to standard prosthesis, particularly for amputees who pursue rigorous or prolonged physical activity.

### RELATED APPLICATIONS

This application claims the benefit under 35 U.S.C. § 119(e) of U.S. Provisional Patent Application No. 62/482,780, filed Apr. 7, 2017, entitled “SHOULDER AND ARM PROSTHESIS,” incorporated herein by reference in entirety.

### BACKGROUND

Medical advances coupled with robotic and prosthetic technology contribute to providing prosthetic limbs as mitigating and rehabilitative remedies in response to traumatic limb loss or compromise. Prosthetic limbs are available to replace all or portions of arms and legs. The human arms play a significant role in stability and efficiency during walking. For shoulder disarticulation and forequarter amputees who have lost an entire arm, these dynamics are no longer a part of their biomechanics. This can lead to detrimental effects on spine health and gait mechanics.

### SUMMARY

The human arm contributes significantly to stability and efficiency during walking. A novel prosthesis is proposed herein that focuses on restoring the complex dynamics of the arm for whole-arm amputees. While most prostheses for arm amputees focus on restoring the user's capabilities for dexterous manipulation, the disclosed prosthesis remains affixed to the shoulder and exerts a moment on the user's trunk similar to that of the arm during walking for dynamic motion assistance. The size, ease of use, and relatively low cost manufacture of the proposed device makes it an attractive complement or alternative to standard prosthesis, especially for amputees who pursue rigorous or prolonged physical activity.

An ambulatory assist device simulates or supplements forces generated in normal human ambulatory movement to provide stability and balance. A control circuit and rotating mass disposed in communication with a wearer detects a gait and stride associated with normal or uninjured movement, and orients the rotating mass in a gimbaled arrangement for generating compensatory moment forces that approximate that which would have been generated by the deficient or missing anatomy. Oscillatory or periodic movement of the gimbaled frame based on the gait therefore effectively simulates the amputee limb or otherwise provides stability through balancing forces. Alternate configurations may include a back or waist mounting for accommodating a coordination loss despite intact limbs, or a leg mounting for lower torso compensation.

Configurations herein are based, in part, on the observation that amputee patients encounter substantial rehabilitation efforts to continue to utilize the remaining limb, as the extremities exhibit complementary forces, the absence of which adversely affects the remaining limbs. Arm movement during ambulatory (walking) activities counteract the movement of the lower body. In the human skeletal frame, arm motion accounts for a 7% increase in metabolic efficiency during walking. Unfortunately, conventional approaches to amputee ambulatory assists tend to be expensive and invasive, including motorized and tethered interventions which may require surgical attachments. Accordingly, configurations herein substantially overcome the above-described shortcomings of conventional cabled or actuated approaches by providing a gyroscopic approach that provides compensatory force to emulate the moment that would otherwise be provided by the compromised arm.

The disclosed compact shoulder prosthesis can restores at least some of the static and dynamic contributions of the human arm. The device is easily customizable to match the weight of the amputee arm, and fits within the form factor of the human shoulder. A defined volume inside the device includes a gyroscopic element that moves in synchronization with the user's stride to exert a moment on the trunk similar to that of the arm during walking. The size, ease of use, and relatively low manufacturing cost of the proposed device makes it an attractive complement or alternative to standard prostheses, especially for amputees who pursue rigorous or prolonged physical activity. These benefits of restored arm dynamics, a balanced torso, and the gyroscopic stabilizing effects of the proposed device can represent a significant improvement to quality of life for arm amputees

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of a shoulder prosthesis suitable for use with the disclosed approach.

FIG. 2 is a perspective view of a frame housing a gyroscopic disc enclosed in the prosthesis of claim 1; and

FIG. 3 is a perspective view of the frame of FIG. 2 in a gimbaled orientation.

### DETAILED DESCRIPTION

Configurations below depict an example prosthesis for detecting and correcting normal balance of a human patient/wearer based on factors such as gait and stride. The disclosed prosthesis implements a gimbaled gyroscopic mass for exerting a moment to simulate, amplify, or assist forces contributing to normal balance and ambulatory patterns. Such a shoulder prosthesis assists arm amputees to regain the dynamic contributions of the arm during walking, running and other movements in a compact form factor that is smaller and less expensive than full arm prostheses. In the prosthetic shoulder example, the moment emulates forces that would be provided by the amputee limb to simulate an uninjured walking motion based on a gait and stride of the wearer. Other configurations may include balance and/or stability assist for compensating for age, skeletal degradation or compromise of nerve control, for example. The prosthesis may be disposed in any suitable location for exerting beneficial moment forces, such as the shoulder, back or leg.

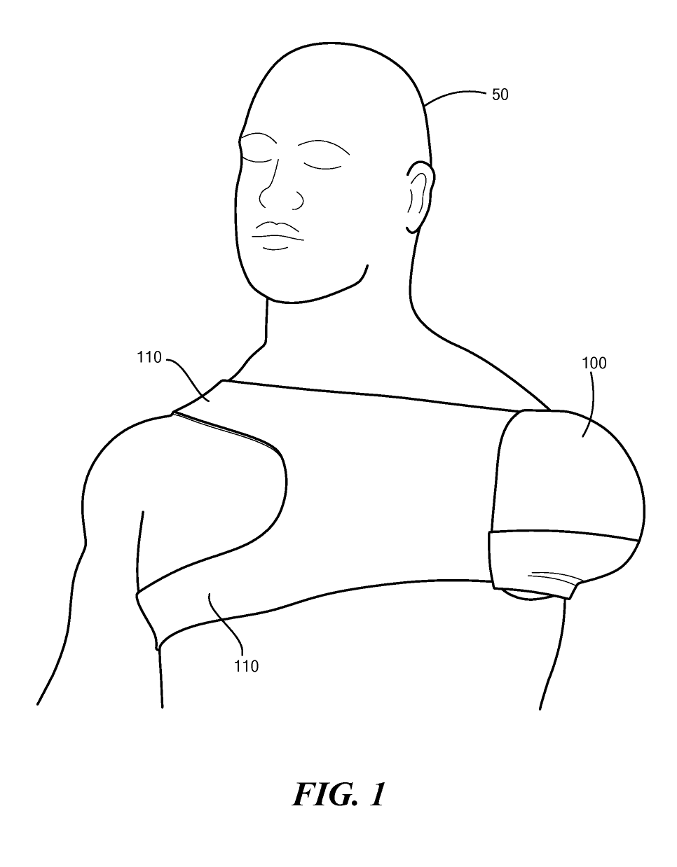

FIG. 1 is a context diagram of a shoulder prosthesis appliance suitable for use with the disclosed approach. The shoulder prosthesis 100 exhibits one example usage of the appliance for exerting moment forces on a human patient or wearer 50 for assisting balance in amputees or coordination challenged circumstances. Moment forces emanate from rotating a mass for generating angular momentum to offset unbalancing forces. An axially controlled spinning mass generates angular momentum for compensating for human balance by controlling an axial orientation of a rotating gyroscopic disk responsive to a detected gait resulting from a normal stride.

The shoulder prosthesis example employs a control moment gyroscope as a gait-assistive tool for arm amputees to replace dynamic contributions of the arm during walking, running, and other forceful or energetic movements based on feedback relating to gist and stride. The method of generating balancing forces responsive to human ambulatory movement includes detecting a normal pattern of movement resulting from ambulatory activity, and detection of off-balance forces indicative of a deviation from the detected normal pattern. A typical appliance includes disposing the rotated mass in a frame secured to a wearer for directing the angular momentum to the wearer. FIG. 1 shows a prosthesis housing 100 including the rotating mass attached to the wearer 50 for transferring the generated angular momentum for achieving postural balance. The prosthesis housing 100 employs a tethered support 110 for securing the frame to a wearer 50 responsive to moment forces generated from the rotating mass. The prosthesis may be worn alone or in combination with prosthesis simulating the amputated limb.

Alternate configurations may deploy the device as an automatic balance device for balance or coordination to prevent falls. The prosthetic device may be positioned more centrally on the back or lower back and it may provide assistive whole body moments to a user to prevent falls. Such a mounting would include a gimbaled frame having a rotating mass, and an attached support for securing the frame to a wearer responsive to moment forces generated from the gimbaled control of the rotating mass. The appliance includes a control circuit operable for rotating the mass for generating angular momentum for offsetting unbalancing forces, and an inertial measurement unit (IMU) for operating the gimbaled frame based on gathered balance forces indicative of upright posture of the wearer.

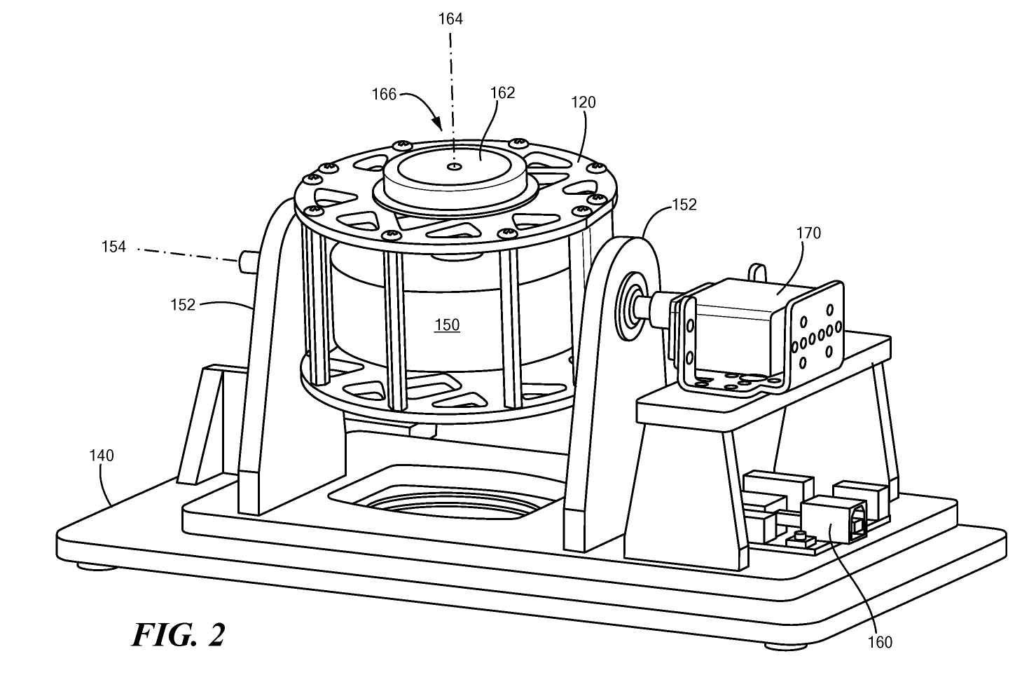

FIG. 2 is a perspective view of a frame 120 housing a gyroscopic disc 150 enclosed in the prosthesis 100 of FIG. 1. The gimbaled frame 120 supports the rotating mass 150 while supported by posts 152 on a gimbal axis 154. A control circuit 160 detects a gait and stride of the wearer 50, such that the rotating mass 150 is responsive to the control circuit 160 for generating angular momentum for compensating for human balance by controlling an axial 154 orientation of a rotating gyroscopic disk from a gimbal motor 170 responsive to a detected gait resulting from a normal stride. The control circuit 160 also controls an axis of rotation 164 of the rotating mass 120 from a “pancake” motor 162 for directing the angular momentum for compensation. The gimbal motor 170 may be a stepper motor or other incrementally adjustable rotary source, and the pancake motor 162 is fulfilled by a compact, high speed drive source suitable for rotating the gyroscopic disc providing the rotating mass 120 (about 1-2 lbs. in the example arrangement).

A base 140 supports the control circuit 160, posts 152, control circuit 160 and gimbal motor 170. The base 140 and accompanying components including the frame 120 are disposed in the shoulder prosthesis 100 adapted to be worn by the wearer 50, such that the frame 120 supports the rotating mass 150 in a gimbaled orientation around a spindle 166 defining the rotation around the axis 164. This assembly allows gimbaling the frame 120 based on the detected gait and stride. The net effect is to rotate the frame 120 along gimbal axis 154 for exerting a moment on the trunk similar to that of the arm during walking by rotating the mass 150 at a speed in the range of 2000-5000 revolutions per minute (RPM) around axis 164.

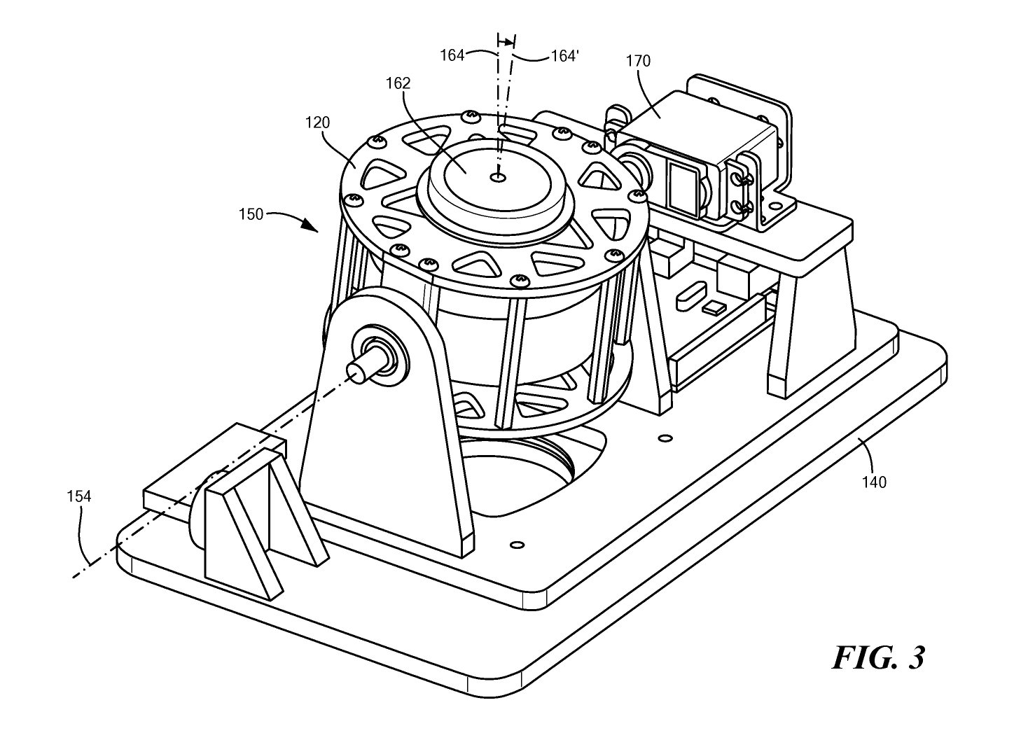

FIG. 3 is a perspective view of the frame of FIG. 2 in a gimbaled orientation. The gimbal motor 170 rotates the gimbaled frame 120 in response to detected off-balance forces, while the pancake motor 162 rotates the mass 150 on the spindle 166 in the frame 120, such that the spindle defines the gimbaled axis 164′. Continuing to refer to FIGS. 2 and 3, gimbaling the mass 120 shifts the axis 164 to the position shown by 164′. Full effect is achieved by rotating the frame 120 in an oscillatory pattern synchronized with a gait of the wearer. In operation, the control circuit 160 detects a speed of the gait and a magnitude of a stride of a wearer 50, and rotates the frame 120 in an oscillatory manner based on the gait and magnitude for approximating forces associated with a natural stride.

In an example prototype using the shoulder, prosthesis configuration includes a 7.6 cm (3 in.) diameter brass 2.5 cm thick (1 in.) disk spinning at 3,000 RPM to create the angular momentum required to exert sufficient arm-like moments on the user. It is expected that an actuator of this size is capable of over 180× torque magnification, creating a 3.6 Nm peak output torque for a 20 mNm input. The actuator will respond to the movements of the user's trunk by using inertial data collected from an IMU also mounted at the shoulder. Control of the device will focus on at least two characteristics: (1) gait frequency, and (2) stride length.

Operation is based on initial IMU data has collected from the shoulder motion of a healthy subject walking at several speeds. Using this data, a the control circuit 160 identifies desired gait characteristics and commands the actuator frequency and magnitude to accurately complement the user's movements.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method of generating balancing forces responsive to human ambulatory movement, comprising:
detecting a normal pattern of movement resulting from ambulatory activity;
detecting off-balance forces indicative of a deviation from the detected normal pattern;
rotating a mass for generating angular momentum for offsetting unbalancing forces; and
controlling an axis of rotation of the rotating mass for directing the angular momentum for compensating.

2. The method of claim 1 further comprising disposing the rotated mass in a frame secured to a wearer for directing the angular momentum to the wearer.

3. The method of claim 1 further comprising attaching a prosthesis housing including the rotating mass to a wearer for transferring the generated angular momentum for achieving postural balance.

4. The method of claim 1 further comprising:
disposing the rotating mass in a gimbaled frame; and
rotating the gimbaled frame in response to the detected off-balance forces.

5. The method of claim 2 further comprising:
rotating the mass on a spindle in a frame, the spindle defining the axis; and
rotating the frame in an oscillatory pattern synchronized with a gait of the wearer.

6. The method of claim 2 further comprising:
detecting a speed of a gait and a magnitude of a stride of a wearer; and
rotating the frame in an oscillatory manner based on the gait and magnitude for approximating forces associated with a natural stride.

7. The method of claim 1 further comprising:
disposing a frame housing the rotating mass in a shoulder prosthesis adapted to be worn by a wearer, the frame supporting the rotating mass in a gimbaled orientation around a spindle defining the rotation; and
rotating the frame for exerting a moment on the trunk similar to that of the arm during walking.

8. The method of claim 1 further comprising rotating the mass at a speed in the range of 2000-5000 revolutions per minute (RPM).

9. The method of claim 1 further comprising gimbaling the frame based on a detected gait and stride.

10. A shoulder prosthesis device, comprising:
a gimbaled frame having a rotating mass;
an attached support for securing the frame to a wearer responsive to moment forces generated from the rotating mass; and
a control circuit for detecting a gait and stride of the wearer, the rotating mass responsive to the control circuit for generating angular momentum for compensating for human balance by controlling an axial orientation of a rotating gyroscopic disk responsive to the detected gait resulting from a normal stride.

11. The device of claim 10 further comprising a tethered support securing the rotating mass and transferring moment forces from the rotating mass to the wearer, the rotating mass disposed in a frame secured to a wearer for directing the angular momentum to the wearer.

12. The device of claim 10 further comprising a prosthesis housing including the rotating mass and adapted for attachment to a wearer for transferring the generated angular momentum for achieving postural balance.

13. The device of claim 10 further comprising:
a gimbaled frame adapted to support the rotating mass for rotation in response to the detected off-balance forces.

14. The device of claim 13 further comprising
a spindle securing the mass in rotational communication with the gimbaled frame, the spindle defining the axis of rotation,
the spindle responsive to rotation of the frame in an oscillatory pattern synchronized with a gait of the wearer.

15. The device of claim 11 further comprising:
a base;
a plurality of posts attached to the base and securing the frame in rotational communication with a gimbal motor for directing moment force based on gait and stride;
a pancake motor attached to the frame and adapted for rotating the mass about the axis of rotation perpendicular to the gimbal axis; and
an inertial measurement unit (IMU) for operating the gimbal motor based on gait and stride.

16. An automatic balance device, comprising:
a gimbaled frame having a rotating mass;
an attached support for securing the frame to a wearer responsive to moment forces generated from the rotating mass;
a control circuit operable for rotating the mass for generating angular momentum for offsetting unbalancing forces; and
an inertial measurement unit (IMU) for operating the gimbaled frame based on gathered balance forces indicative of upright posture of the wearer.
